(19) United States
(12) Patent Application Publication
(10) Pub. No.: US 2021/0076938 A1
(43) Pub. Date: Mar. 18, 2021

(54) HYBRID SYSTEM FOR PERFORMING A MAGNETIC RESONANCE TOMOGRAPHY AND A RADIOFREQUENCY ABLATION, AND METHOD FOR THE OPERATION THEREOF

(71) Applicant: OTTO-VON-GUERICKE-UNIVERSITÄT MAGDEBURG, Magdeburg (DE)

(72) Inventors: Thomas GERLACH, Magdeburg (DE); Enrico PANNICKE, Magdeburg (DE)

(21) Appl. No.: 16/968,300

(22) PCT Filed: Feb. 6, 2019
(86) PCT No.: PCT/EP2019/052896

§ 371 Date: Aug. 7, 2020

(30) Foreign Application Priority Data
Feb. 9, 2018 (DE) 10 2018 102 965.9

(51) Int. Cl. A61B 5/00; A61B 5/055; A61B 18/12; A61B 18/14

(52) U.S. Cl. CPC A61B 5/0036; A61B 5/055; A61B 2018/00577; A61B 18/14; A61B 18/1206

(57) ABSTRACT

The invention relates to a hybrid system for performing a magnetic resonance tomography (MRT) and a radiofrequency ablation on a patient, comprising the following characteristics: a) the hybrid system comprises a magnetic resonance tomography system in which MRT high-frequency signals for carrying out the magnetic resonance tomography can be generated and supplied on an output terminal of the magnetic resonance tomography system; b) the hybrid system comprises at least one ablation electrode for performing the radiofrequency ablation; and c) the at least one ablation electrode is coupled to the output terminal of the magnetic resonance tomography system such that the radiofrequency ablation can be carried out by the at least one ablation electrode by means of the MRT high-frequency signals. The invention also relates to a method for operating such a hybrid system.

The invention relates to a hybrid system for carrying out magnetic resonance imaging (MRI) and radiofrequency ablation on a patient. Moreover, the invention relates to a method for operating such a hybrid system.

Magnetic resonance imaging, abbreviated MRI, is an imaging method used in medicine for presenting structure and function of tissue and organs in the body. Using MRI, it is possible to generate slice images of the body of a patient. To carry out the MRI, radiofrequency signals are generated by the magnetic resonance imaging system, said radiofrequency signals being referred to as MRI radiofrequency signals below. These MRI radiofrequency signals are fed into the patient in the form of alternating magnetic fields with a high amplitude, for example via a transmit coil. At a certain frequency (the so-called Larmor frequency), this leads to certain atomic nuclei being resonantly excited in the body, as a result of which an electrically induced signal can be measured in a receiver circuit following the deactivation of the RF field.

The procedure of transmitting RF fields and receiving measurement signals is repeated with the activation of separate spatially dependent magnetic fields. This allows spatial encoding of the signals in the body. The recorded signals can then be evaluated and visualized in computer-assisted post-processing.

Radiofrequency ablation, abbreviated RFA, is a hyperthermal and minimally invasive approach for destroying tumors and metastases. As a rule, radiofrequency ablation is carried out using medical imaging, for the purposes of which magnetic resonance imaging is preferably used. In radiofrequency ablation, a radiofrequency generator is coupled via shielded cables to an ablation electrode, by means of which the radiofrequency energy is fed into the patient. This often requires great outlay to avoid disadvantageous influencing of the MR imaging by the radiofrequency ablation. Overall, this requires great outlay in terms of equipment.

The invention is based on the object of reducing the outlay for MRI-guided radiofrequency ablation.

This object is achieved by a hybrid system for carrying out magnetic resonance imaging (MRI) and radiofrequency ablation on a patient, including the following features:
a) the hybrid system comprises a magnetic resonance imaging system, in which MRI radiofrequency signals for carrying out magnetic resonance imaging are generable and providable at an output connector of the magnetic resonance imaging system,
b) the hybrid system comprises at least one ablation electrode for carrying out radiofrequency ablation,
c) the at least one ablation electrode is coupled to the output connector of the magnetic resonance imaging system such that radiofrequency ablation is able to be carried out by way of the at least one ablation electrode by means of the MRI radiofrequency signals.

It was discovered that the MRI radiofrequency signals provided by the magnetic resonance imaging system, which are used for carrying out the magnetic resonance imaging examination and the imaging therefor, are also suitable for carrying out the radiofrequency ablation. In particular, the MRI radiofrequency signal energy provided at the output of an RF amplifier of the magnetic resonance imaging system, for example, is sufficient for carrying out radiofrequency ablation. Consequently, the entire system can be simplified since there is no need for a separate radiofrequency generator for feeding the ablation electrode. Instead, the ablation electrode or, where necessary, a plurality of ablation electrodes can be connected to the output connector of the magnetic resonance imaging system, either directly or via additional components. In this way, the at least one ablation electrode is fed with the radiofrequency energy of the MRI radiofrequency signals such that the radiofrequency ablation can be carried out directly by means of the MRI radiofrequency signals.

This simplifies the entire system, costs can be saved and, moreover, fewer components that have to be tested or that could fail or cause disturbances are required. Moreover, disturbing influences of the radiofrequency ablation can be reduced during MR imaging since separate signals, which are not synchronized with the MRI radiofrequency signals, are not fed into the patient.

According to an advantageous development of the invention, provision is made for the hybrid system to comprise a pulse generation circuit, by means of which the radiofrequency signals required for radiofrequency ablation are suppliable in pulsed fashion to the at least one ablation electrode. This allows pulsed radiofrequency ablation to be carried out. Particularly in combination with the use of the MRI radiofrequency signals for the radiofrequency ablation, this yields the advantageous synergy effect that the MRI radiofrequency signals are already generated in pulsed fashion in conventional magnetic resonance imaging systems, e.g., in the form of pulse trains consisting of a multiplicity of individual radiofrequency pulses, with a pause, during which no radiofrequency pulses are generated, being present between such pulse trains.

According to an advantageous development of the invention, provision is made for the at least one ablation electrode to be fed with a radiofrequency signal at the Larmor frequency. This is advantageous in that the ablation current fed into the patient by the ablation electrode generates a magnetic eddy current field which, in turn, generates measurable magnetic resonance signals such that this eddy current field can also be captured and visualized by the MR imaging unit.

According to an advantageous development of the invention, provision is made for the hybrid system to be configured to record and visualize the ablation current, which is fed into the patient by the ablation electrode, by way of the imaging unit of the magnetic resonance imaging system. This is advantageous in that the user of the hybrid system can be provided with additional information about the current state of the radiofrequency ablation. By way of example, the signal intensities arising during the MR imaging can supply qualitative information about the current profile of the ablation current. For the purposes of visualizing current, it is possible, for example, to carry out a measurement of the amplitude and the phase of the magnetic fields, from which it is possible to reconstruct the amplitude and phase of the ablation current. Moreover, by using the MRI radiofrequency signals for the radiofrequency ablation, there is no need for complicated phase synchronization between the ablation signal and the MR imaging.

According to an advantageous development of the invention, provision is made for the hybrid system to be configured to supply the MRI radiofrequency signals either to the at least one ablation electrode or to an MRI transmit coil of the magnetic resonance imaging system. This avoids mixed use of the individual signal pulses of the MRI radiofrequency signals. Instead, the MRI radiofrequency signals are always only supplied to one use at any one time, i.e., either to the radiofrequency ablation or to the imaging within the scope of magnetic resonance imaging. This can ensure a high image quality for magnetic resonance imaging. To supply the MRI radiofrequency signals either to the ablation electrode or to the MRI transmit coil, a computer-controlled changeover switch, for example, can be present.

The object set forth at the outset is also achieved by a method for operating a hybrid system of the aforementioned type, in which the MRI radiofrequency signals of the magnetic resonance imaging system are supplied to the at least one ablation electrode at least intermittently. This also allows the aforementioned advantages to be realized.

According to an advantageous development of the invention, provision is made for the MRI radiofrequency signals of the magnetic resonance imaging system to be alternately supplied to the at least one ablation electrode and an MRI transmit coil of the magnetic resonance imaging system. This allows an undisturbed signal with a full signal intensity to be used either for the one application or for the other application in each case. This is beneficial to the quality of the MR imaging, in particular.

According to an advantageous development of the invention, provision is made for the image generation for visualizing the magnetic resonance imaging examination to be interrupted while the at least one ablation electrode is fed with the MRI radiofrequency signals. This interruption of the image generation does not bother the user since it is so short-term that it is substantially not perceived.

According to an advantageous development of the invention, provision is made for the magnetic eddy current field generated in the patient by the ablation current of the at least one ablation electrode to be recorded by way of the magnetic resonance imaging system and visualized as a current profile. This can supply the user with additional information about the current state of the radiofrequency ablation.

The invention will be explained in more detail below on the basis of exemplary embodiments using drawings.

In the drawings:

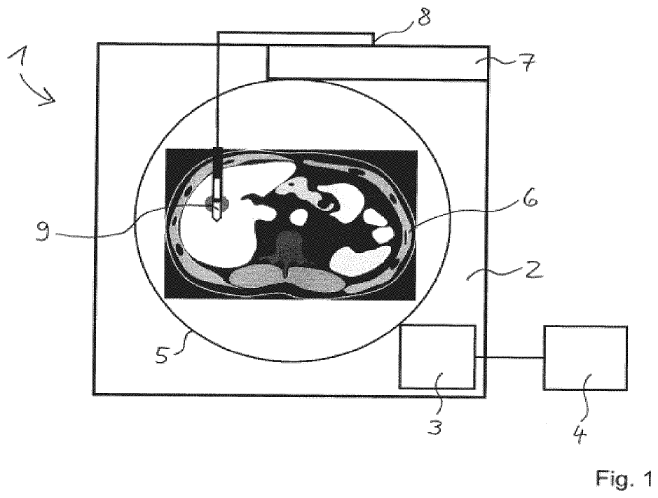

FIG. 1 shows a schematic illustration of a hybrid system and

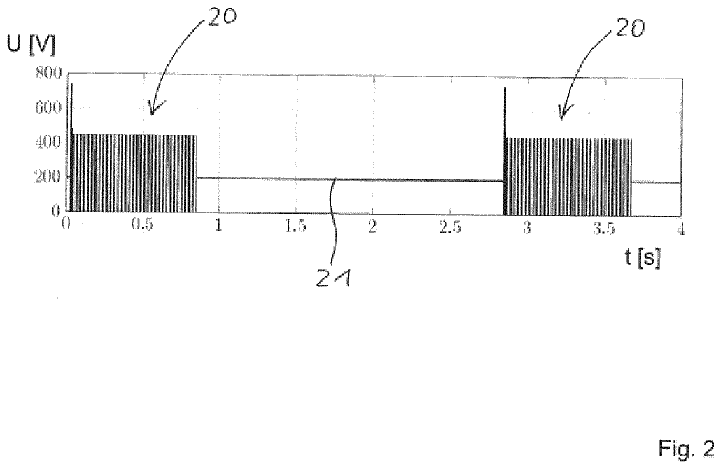

FIG. 2 shows the generation of pulsed radiofrequency signals and

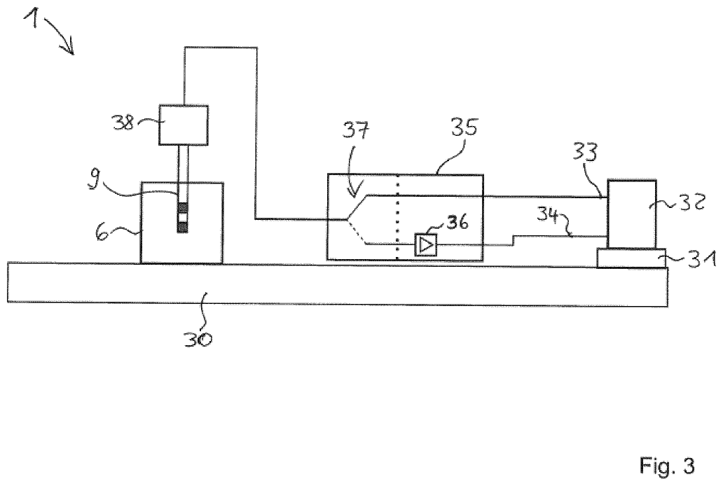

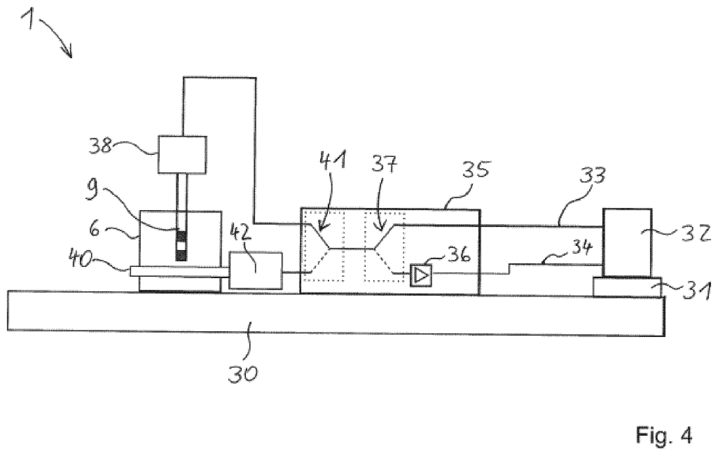

FIGS. 3 and 4 show further embodiments of a hybrid system.

FIG. 1 shows a hybrid system 1 comprising a magnetic resonance imaging system 2. The magnetic resonance imaging system 2 can be of a conventional, known design. By way of example, the magnetic resonance imaging system 2 comprises a tube 5 to be used for magnetic resonance imaging examinations, into which a patient 6 can be placed. The MRI radiofrequency signals provided by a radiofrequency amplifier 7 of the magnetic resonance imaging system 2 are transferred to the patient 6 by way of transmit coils, which are arranged, for example, in the wall of the tube 5. The resultant signals of the magnetic resonance imaging system, recorded on the receiver side, are captured and processed by way of an imaging unit 3 of the magnetic resonance imaging system 2. The image information generated thereby can be presented on an image display device 4.

The hybrid system 1 is further configured to carry out a radiofrequency ablation on the patient 6. To this end, at least one ablation electrode 9 is present, which can be placed, for example, against a tumor to be removed within the patient 6. The ablation electrode 9 is connected to an output connector 8 of the magnetic resonance imaging system, e.g., an output connector of the RF output amplifier 7, via a line. In this way, the MRI radiofrequency signals provided at the output connector 8 are supplied to the ablation electrode 9 and fed into the patient 6.

FIG. 2 shows an exemplary time profile of the MRI radiofrequency signals 20. The MRI radiofrequency signals 20 can be generated in the form of individual pulse trains, of which two pulse trains are illustrated in FIG. 2. A pulse train consists of a multiplicity of individual radiofrequency pulses. There is a pause, e.g., a pause of approximately 2 seconds in the illustrated example, between the individual pulse trains. The line 21 represents a mean voltage, which is established as effective voltage for the ablation process on the ablation electrode 9. By way of example, the first pulse train 20 illustrated in FIG. 2 can be used for radiofrequency ablation and consequently only be supplied to the ablation electrode and the other, second illustrated pulse train 20 can be used for imaging within the scope of magnetic resonance imaging, i.e., this pulse train is only supplied to an MRI transmit coil.

FIG. 3 shows further features of the hybrid system 1, which can be realized, for example, in the hybrid system explained on the basis of FIG. 1. A patient couch 30, on which the patient 6 is placed, is identifiable. Further, the ablation electrode 9 is illustrated once again. The ablation electrode 9 is connected to a function block 35 by way of an interface circuit 38. The function block 35 contains a changeover switch 37, e.g., in the form of an RX-TX switch. By way of the changeover switch 37, the ablation electrode 9 can alternatively be connected to a transmitter channel or receiver channel of the hybrid system 1. The transmitter channel can be connected to the function block 35 via a transmitter line 33, the receiver channel via a receiver line 34. The function block 35 comprises a preamplifier 36 for the receiver channel, said preamplifier being connected to the receiver line 34. The transmitter line 33 and the receiver line 34 are connected to a coil plug 32, to which the transmitter channel and the receiver channel of the hybrid system 1 can be connected. The coil plug 32 can be connected to a coil terminal 31, from which the radiofrequency signals for imaging or ablation can be taken.

During the operation of the hybrid system as per FIG. 3, the changeover switch 37 is changed over in computer-controlled fashion, for example alternately in the case of the pulse trains illustrated in FIG. 2, such that the MRI radiofrequency signals are alternately supplied to the one or the other application, and so the ablation electrode 9 alternately acts in the transmission case or in the reception case.

FIG. 4 shows a further configuration of the hybrid system 1, which differs from the embodiment of FIG. 3 as follows: An MRI coil 40, e.g., in the form of a conductor loop, is present for MR imaging or for recording the magnetic fields to carry out MR imaging. The MRI coil 40 is connected to the function block 35 by way of an interface circuit 42. In addition to the aforementioned changeover switch 37 and the preamplifier 36, the function block 35 additionally comprises a further changeover switch 41.

During the operation of the hybrid system as per FIG. 4, the changeover switch 41 is changed over in computer-controlled fashion, for example alternately in the case of the pulse trains illustrated in FIG. 2, such that the MRI radiofrequency signals are alternately supplied to the ablation electrode 9 or the MRI coil 40.

The ablation application is possible when the changeover switch 41 is switched to the ablation electrode 9 and the MR imaging application is possible when said changeover switch is switched to the MRI coil 40.

1. A hybrid system for carrying out magnetic resonance imaging (MRI) and radiofrequency ablation on a patient (6), comprising:
a magnetic resonance imaging system in which MRI radiofrequency signals for carrying out magnetic resonance imaging are generatable and providable at an output connector of the magnetic resonance imaging system,
at least one ablation electrode for carrying out radiofrequency ablation,
wherein the at least one ablation electrode is coupled to the output connector of the magnetic resonance imaging system such that radiofrequency ablation is able to be carried out by way of the at least one ablation electrode using the MRI radiofrequency signals.

2. The hybrid system as claimed in claim 1, further comprising a pulse generation circuit for supplying the MRI radiofrequency signals to the at least one ablation electrode in pulsed fashion.

3. The hybrid system as claimed in claim 1 wherein the at least one ablation electrode is fed with a radiofrequency signal from the MRI radiofrequency signals that is at the Larmor frequency.

4. The hybrid system as claimed in claim 1 further comprising an imaging unit associated with magnetic resonance imaging system, wherein the imaging unit is configured to record and visualize an ablation current which is fed into a patient by the at least one ablation electrode.

5. The hybrid system as claimed in claim 1 wherein hybrid system is configured to supply the MRI radiofrequency signals either to the at least one ablation electrode or to an MRI transmit coil of the magnetic resonance imaging system.

6. A method for operating a hybrid system as claimed in claim 1 comprising supplying the MRI radiofrequency signals of the magnetic resonance imaging system to the at least one ablation electrode intermittently.

7. The method as claimed in claim 6, wherein the MRI radiofrequency signals of the magnetic resonance imaging system are alternately supplied to the at least one ablation electrode and an MRI transmit coil of the magnetic resonance imaging system.

8. The method as claimed in claim 6, further comprising interrupting generation an image for visualizing the magnetic resonance imaging examination while the at least one ablation electrode is fed with the MRI radiofrequency signals.

9. The method as claimed in claim 6 further comprising recording a magnetic eddy current field generated in a patient by an ablation current of the at least one ablation electrode by the magnetic resonance imaging system; and visualizing a recorded magnetic eddy current field as a current profile.
